(19) United States
(12) Patent Application Publication
(10) Pub. No.: US 2018/0271435 A1
(43) Pub. Date: Sep. 27, 2018

(54) LEARNING SLEEP STAGES FROM RADIO SIGNALS

(71) Applicant: Massachusetts Institute of Technology, Cambridge, MA (US)

(72) Inventors: Mingmin Zhao, Cambridge, MA (US); Shichao Yue, Cambridge, MA (US); Dina Katabi, Cambridge, MA (US); Tommi S. Jaakkola, Burlington, MA (US)

(21) Appl. No.: 15/933,921

(22) Filed: Mar. 23, 2018

Related U.S. Application Data
(60) Provisional application No. 62/476,815, filed on Mar. 26, 2017.
(60) Provisional application No. 62/518,053, filed on Jun. 12, 2017.

(51) Int. Cl. A61B 5/00; A61B 5/04; G06N 3/02; G06N 5/04

(52) U.S. Cl. CPC A61B 5/4812; G06N 5/046; G06N 3/02; A61B 5/04

(57) ABSTRACT

A method for tracking a sleep stage of a subject takes as input a sequence of observations sensed over an observation time period. The sequence of observation values is processed to yield a corresponding sequence of encoded observations using a first artificial neural network (ANN) and the sequence of encoded observation values is processed to yield a sequence of sleep stage indicators using a second artificial network. Each observation may correspond to an interval of the observation period (e.g., at least 30 seconds). The first ANN may be configured to reduce information representing a source of the sequence of observations in the encoded observations.

### CROSS-REFERENCE TO RELATED APPLICATIONS

This application claims the benefit of U.S. Provisional Application No. 62/476,815, filed on Mar. 26, 2017, titled “Learning Sleep Stages from Radio Signals,” and U.S. Provisional Application No. 62/518,053, filed on Jun. 12, 2017, titled “Learning Sleep Stages from Radio Signals,” which is incorporated herein by reference. This application is also related to U.S. Pat. Pub. 2017/0042432, titled “Vital Signs Monitoring Via Radio Reflections,” and to U.S. Pat. No. 9,753,131, titled “Motion Tracking Via Body Radio Reflections,” which are also incorporated herein by reference.

### BACKGROUND

This invention relates to inference of sleep stages of a subject via radio signals.

Sleep plays a vital role in an individual's health and well-being. Sleep progresses in cycles that involve multiple sleep stages: Awake, Light sleep, Deep sleep and REM (Rapid eye movement). Different stages are associated with different physiological functions. For example, deep sleep is essential for tissue growth, muscle repair, and memory consolidation, while REM helps procedural memory and emotional health. At least, 40 million Americans each year suffer from chronic sleep disorders. Most sleep disorders can be managed once they are correctly diagnosed. Monitoring sleep stages is critical for diagnosing sleep disorders, and tracking the response to treatment.

Prevailing approaches for monitoring sleep stages are generally inconvenient and intrusive. The medical gold standard relies on Polysomnography (PSG), which is typically conducted in a hospital or sleep lab, and requires the subject to wear a plethora of sensors, such as EEG-scalp electrodes, an ECG monitor, and a chest band or nasal probe for monitoring breathing. As a result, patients can experience sleeping difficulties which renders the measurements unrepresentative. Furthermore, the cost and discomfort of PSG limit the potential for long term sleep studies.

Recent advances in wireless systems have demonstrated that radio technologies can capture physiological signals without body contact. These technologies transmit a low power radio signal (i.e., 1000 times lower power than a cell phone transmission) and analyze its reflections. They extract a person's breathing and heart beats from the radio frequency (RF) signal reflected off her body. Since the cardio-respiratory signals are correlated with sleep stages, in principle, one could hope to learn a subject's sleep stages by analyzing the RF signal reflected off her body. Such a system would significantly reduce the cost and discomfort of today's sleep staging, and allow for long term sleep stage monitoring.

There are multiple challenges in realizing the potential of RF measurements for sleep staging. In particular, RF signal features that capture the sleep stages and their temporal progression must be learned, and such features should be transferable to new subjects and different environments. A problem is that RF signals carry much information that is irrelevant to sleep staging, and are highly dependent on the individuals and the measurement conditions. Specifically, they reflect off all objects in the environment including walls and furniture, and are affected by the subject's position and distance from the radio device. These challenges were not addressed in past work which used hand-crafted signal features to train a classifier. The accuracy was relatively low (about 64%) and the model did not generalize beyond the single environment where the measurements were collected.

Recent advances in use of Convolutional Neural Networks (CNN) and Recurrent Neural Networks (RNN) have led to successful use to model spatial patterns and temporal dynamics. Generative Adversarial Networks (GAN) and their variants have been used to model mappings from simple latent distributions to complex data distributions. Those learned mappings can be used to synthesize new samples and provide semantically meaningful arithmetic operations in the latent space. Bidirectional mapping has also been proposed to learn the inverse mapping for discrimination tasks.

### SUMMARY

In one aspect, in general, a method for tracking a sleep stage of a subject takes as input a sequence of observation values (xᵢ), which may be referred to as “observations” for short, sensed over an observation time period. The sequence of observation values is processed to yield a corresponding sequence of encoded observation values (zᵢ), which may be referred to as “encoded observations” for short. The processing of the sequence of observation values includes using a first artificial neural network (ANN) to process a first observation value to yield a first encoded observation value. The sequence of encoded observation values is processed to yield a sequence of sleep stage indicators (ŷᵢ, or Q(y|zᵢ)) representing sleep stage of the subject over the observation time period. This includes processing a plurality of the encoded observation values, which includes the first encoded observation value, using a second artificial network (ANN), to yield a first sleep stage indicator.

Aspects of the method for tracking sleep stage may include one or more of the following features.

Each observation corresponds to at least a 30 second interval of the observation period.

The first ANN is configured to reduce information representing a source of the sequence of observations in the encoded observations.

The first ANN comprises a convolutional neural network (CNN), and the second ANN comprises a recurrent neural network (RNN).

The sequence of sleep stage indicators includes a sequence of inferred sleep stages (ŷᵢ) from a predetermined set of sleep stages, and/or includes a sequence of probability distributions of sleep stage across the predetermined set of sleep stages.

Determining the sequence of observations (xᵢ) includes acquiring a signal including at least a component representing the subject's breathing, and processing the acquired signal to produce the sequence of observations such that the observations in the sequence represent variation in the subject's breathing.

Acquiring the sequence of observation values includes emitting a radio frequency reference signal, receiving a received signal that includes a reflected signal comprising a reflection of the reference signal from the body of the subject, and processing the received signal to yield an observation value representing motion of the body of the subject during a time interval within the observation time period.

The time interval for each observation value is at least 30 seconds in duration.

Processing the received signal includes selecting a component of the received signal corresponding to a physical region associated with the subject, and processing the component to represent motion substantially within that physical region.

Acquiring the sequence of observation values comprises acquiring signals from sensors affixed to the subject.

In another aspect, in general, a method for tracking a sleep stage of a subject includes acquiring a sequence of observation values (xᵢ) by sensing the subject over an observation time period. The sequence of observation values is processed to yield a corresponding sequence of encoded observation values (zᵢ). The processing of the sequence of observation values includes using a first parameterized transformation (e.g., a first ANN, for example a convolutional network), configured with values of a first set of parameters (θₑ), to process a first observation value to yield a first encoded observation value. The sequence of encoded observation values is processed to yield a sequence of sleep stage indicators (Q(y|zᵢ)) representing sleep stage of the subject over the time period, including processing a plurality of encoded observation values, which includes the first encoded observation value, using a second parameterized transformation, configured with values of a second set of parameters (θ_{f}), to yield a first sleep stage indicator.

The method can further include determining the firsts set of parameter values and the second set of parameter values by processing reference data that represents a plurality of associations (tuples), each association including an observation value (xᵢ), a corresponding sleep stage (yᵢ), and a corresponding source value (sᵢ). The processing determines values of the first set of parameters to optimize a criterion (ν) to increase information in the encoded observation values, determined from an observation value according to the values of the first set of parameters, related to corresponding sleep stages, and to reduce information in the encoded observation values related to corresponding source values.

The processing of the reference data that represents a plurality of associations further may include determining values of a third set of parameters (θ_{d}) associated with a third parameterized transformation, third parameterized transformation being configured to process an encoded observation value to yield and indicator of a source value (Q(s|zᵢ) ). For example, the processing of the reference data determines values of the first set of parameters, values of the second set of parameters, and values of the third set of parameters to optimize the criterion. In some examples, the information in the encoded observation values related to corresponding sleep stages depends on the values of the second set of parameter and information in the encoded observation values related to corresponding source values depends on the values of the third set of parameters.

In another aspect, in general, a machine-readable medium comprising instructions stored thereon, which when executed by a processor cause the processor to perform the steps of any of the methods disclosed above.

In another aspect, in general, a sleep tracker is configured to perform the steps of any of the methods disclosed above.

In yet another aspect, in general, a training approach for data other than sleep related data makes use of tuples of input, output, and source values. A predictor of the output from the input includes an encoder, which produced encoded inputs, and a predictor that takes encoded input and yields a predicted output. Generally, parameters of the encoder are selected (e.g., trained) to increase information in the encoded inputs related to corresponding true output, and to reduce information in the encoded input related to corresponding source values.

An advantage of one or more of the aspects outlined above or described in detail below is that the predicted output (e.g., predicted sleep stage) has high accuracy, and in particular is robust to difference between subjects and to difference is signal acquisition conditions.

Another advantage of one or more aspects is an improved insensitivity to variations in the source of the observations rather than features of the observations that represent the sleep state. In particular, the encoder of the observations may be configured in an unconventional manner to reduce information representing a source of the sequence of observations in the encoded observations. A particular way of configuring the encoder is to determine parameters of an artificial neural network implementing the encoder using a new technique referred to below as “conditional adversarial training.” It should be understood that similar approaches may be applied to other types of parameterized encoders than artificial neural networks. Generally, the parameters of the encoder may be determined according to an optimization criterion that both preserves the desired aspects of the observations, for example, preserving the information that helps predict sleep stage, while reducing information about undesired aspects, for example, that represent the source of the observations, such as the identity of the subject or the signal acquisition setup (e.g., the location, modes of signal acquisition, etc.).

Other aspects and advantage are evident from the description below, and from the claims.

### DESCRIPTION OF DRAWINGS

FIG. 1 is a block diagram of a runtime sleep stage processing system.

FIG. 2 is a block diagram of a parameter estimation system for the runtime system of FIG. 1.

FIG. 3 is an artificial neural network (ANN) implement of a sleep tracker.

FIGS. 4-6 are a block diagrams of training systems; and

FIG. 7 is a block diagram of a reflected radio wave acquisition system.

### DETAILED DESCRIPTION

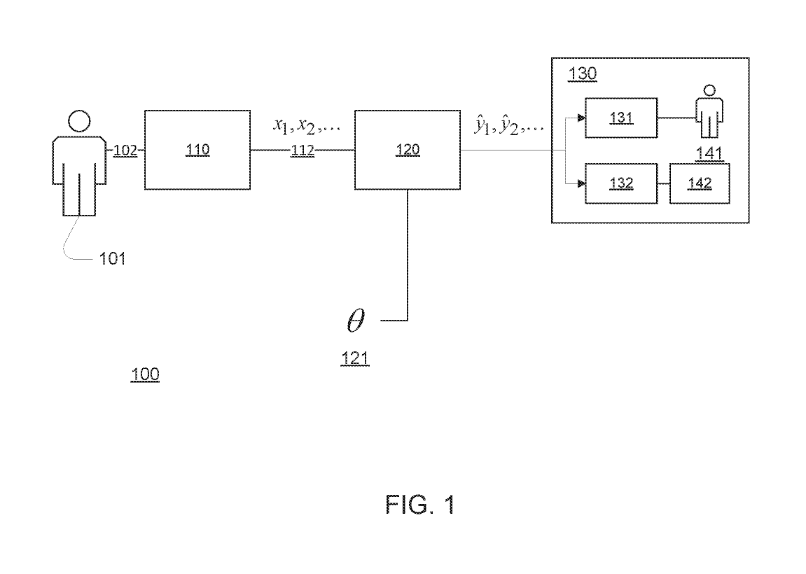

Referring to FIG. 1, a sleep stage processing system 100 monitors a subject 101, who is sleeping, and infers the stage of the subject's sleep as a function of time. Various sets of predefined classes can be used in classifying sleep stage. For example, the stages may include a predetermined enumeration including but not limited to a four-way categorization: “awake,” “light sleep,” “deep sleep,” and “Rapid Eye Movement (REM).” The system 100 includes a signal acquisition system 110, which processes an input signal 102 which represents the subject's activity, for example, sensing the subject's breath or other motion. As discussed further below, the signal acquisition system 110 may use a variety of contact or non-contact approaches to sense the subject's activity by acquiring one or more signals or signal components that represent the subject's respiration, heartrate, or both, for example, representing the subject's motion induced by the subject's breathing and heartbeat. In one embodiment, the system may use reflected radio waves to sense the subject's motion, while in other embodiments the system may use an electrical sensor signal (e.g., a chest-affixed EKG monitor) coupled to the subject.

The output of the signal acquisition module 110 is a series of observation values 112, for instance with one observation value produced every 30 seconds over an observation period, for example spanning may hours. In some cases, each observation value represents samples of a series of acquired sample values, for example, with samples every 20 ms. and one observation value 112 represents a windowed time range of the sample values. In the description below, an observation value at a time index i is denoted xᵢ (i.e., a sequence or set of sample values for a single time index). Below, xᵢ (boldface) denotes the sequence of observation values, xᵢ=(x₁, x₂, . . . , xᵢ), ending at the current time i, and in the case of a missing subscript, x represents the sequence up to the current time index.

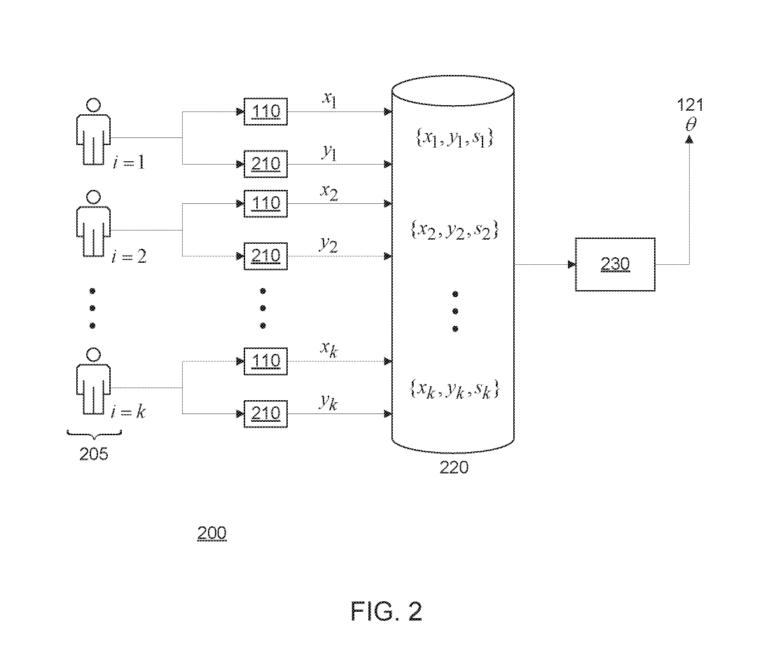

The series of observation values 112 passes to a sleep stage tracker 120, which processes the series and produces a series of an inferred sleep stages 122 for corresponding time indexes i, denoted as ŷᵢ, based on the series of observation values xᵢ. Each value ŷᵢ belongs to the predetermined set of sleep stages, and is an example of a sleep stage indicator. The sleep stage tracker 120 is configured with values of a set of parameters, denoted θ 121, which controls the transformation of the sequence of observation values 112 to the sequence of inferred sleet stages 122. Approaches to determining these parameter values are discussed below with reference to FIG. 2.

The series 122 of inferred sleep stages may be used by one or more end systems 130. For instance, a notification system 131 monitors the subject's sleep stage and notifies a clinician 140, for example, when the subject enters a light sleep stage and may wake up. As another example, a prognosis system 132 may process the sleep stage to provide a diagnosis report based on the current sleep stage sequence, or based on changes in the pattern of sleep stages over many days.

Referring to FIG. 2 a configuration system 200 of the sleep processing system 100 of FIG. 1 is used to determine the values of a set of parameters θ 121 used by at runtime by the sleep processing system 100. The configuration system uses a data set 220 collected from a set of subjects 205. For each of the training subjects, corresponding observation values xᵢ and known sleep stages yᵢ are collected. For example, the observation values xᵢ are produced using a signal acquisition module 110 of the same type as used in the runtime system, and the known sleep stages yᵢ are determined by a process 210, for example, by manual annotation, or based on some other monitoring of the subject (e.g., using EEG data). Note that the sleep stages yᵢ used in the configuration are treated as being “truth”, while the inferred sleep stages ŷᵢ produced by the sleep tracker 120 of FIG. 1 are inferred estimates of those sleep stages, which would ideally be the same, but more generally will deviate from the “true” stages. The data from each subject is associated with a source identifier from an enumerated set of sources. A source value for each observation xᵢ and stage yᵢ is recorded and denoted sᵢ. Therefore, the data used for determining the parameters consists of (or is stored in a manner equivalent to) a set of associations (tuples, triples) comprising (xᵢ, yᵢ, sᵢ), where sᵢ denotes the source (e.g., an index of the training subject and/or the recording environment) corresponding the observation value xᵢ and true sleep stage yᵢ.

Once the system gathers the data set 220, a parameter estimation system 230 processes the training data to produce the values of parameters θ 121. Generally, the system 230 processes the tuples with a goal that the sleep stage tracker 120 (shown in FIG. 1) configured with θ will track sleep stage on new subjects in previously unseen environments by discarding all extraneous information specific to externalities (e.g., the specific training subject from whom a given tuple is derived, measurement conditions) so as to be left with sleep-specific subject-invariant features from input signals. The purpose of discarding such information is to enhance the system's ability to function for a wide range of subjects and a wide range of data acquisition methods.

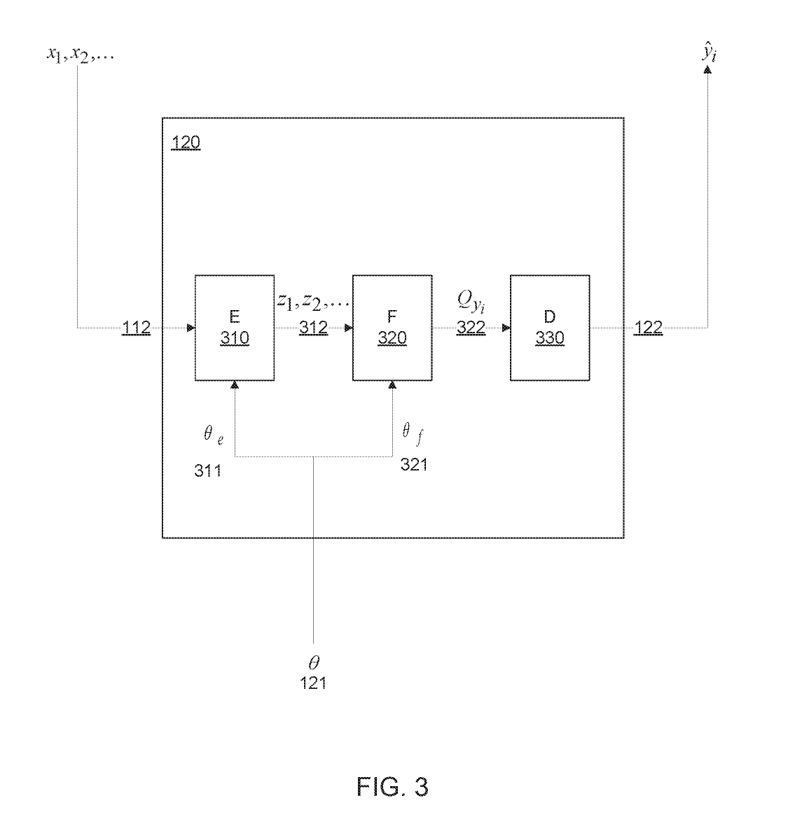

Referring to FIG. 3, the sleep stage tracker 120 includes multiple sequential stages of processes, labelled E (310), F (320), and M (330). Very generally, stage E 310 implements a “encoder” that takes a current observation value (xᵢ) or more generally sequence of observation values xᵢ and outputs an encoded observation value (“encoding”) zᵢ=E(xᵢ) of those observation values. Stage F 320 implements a “label predictor” that processes a current encoding (zᵢ), or more generally sequence of observation values zᵢ=(z₁, . . . , zᵢ), and outputs a probability distribution over the possible sleep stages y, denoted Q_{F}(y|zᵢ), which can also be considered to be a sleep stage indicator. Finally, stage M 330 implements a “label selector” that processes the distribution of the sleep stage, outputs a selected “best” sleep stage ŷᵢ.

In one embodiment, stage E 310 is implemented as a convolutional neural network (CNN) that is configured to extract sleep stage specific data from a sequence of observation values 112, while discarding information that is may encode the source or recording condition. In some embodiments, this sequence of observation values 112 may be presented to encoder E 310 as RF spectrograms. In this embodiment, each observation value xᵢ represents an RF spectrogram of the 30 second window. Specifically, the observation value includes an array with 50 samples per second and 10 frequency bins, for an array with 1,500 time indexes by 10 frequency indexes producing a total of 15,000 complex scalar values, or 30,000 real values with each complex value represented as either a real and imaginary part or as a magnitude and phase. The output of the encoder is a vector scalar values. The CNN of the encoder E 310 is configured with weights that are collectively denoted as θₑ 311, which is a subset of parameter variable θ 121.

In some embodiments, the label predictor F 320 is implemented as a recurrent neural network (RNN). The label predictor 320 takes as input the sequence of encoded values zᵢ 312 and outputs the predicted probabilities over sleep stage labels yᵢ. In this embodiment, the number of outputs of the label predictor 320 is the number of possible sleep stages, with each output providing a real value between 0.0 and 1.0, with the sum of the outputs constrained to be 1.0, representing a probability of that sleep stage. The recurrent nature of the neural network maintains internal stage (i.e., values that are fed back from an output at one time to the input at a next time) and therefore although successive encoded values zᵢ are provided as input, the output distribution depends on the entire sequence of encoded values zᵢ. Together, the cascaded arrangement of E 310 and F 320 can be considered to compute a probability distribution Q_{F}(y|xᵢ). The label predictor F 320 is configured by a set of parameters θ_{f}, which is a subset of parameter variable θ 121.

In some embodiments, stage M 330 is implemented as a selector that determines the value ŷᵢ that maximizes Q_{F}(y|zᵢ) over sleep stages y. In this embodiment, the selector 330 is not parameterize. In other embodiments the stage M may smooth, filter, track or otherwise process the outputs of the label predictor to estimate or determine the evolution of the sleep stage over time.

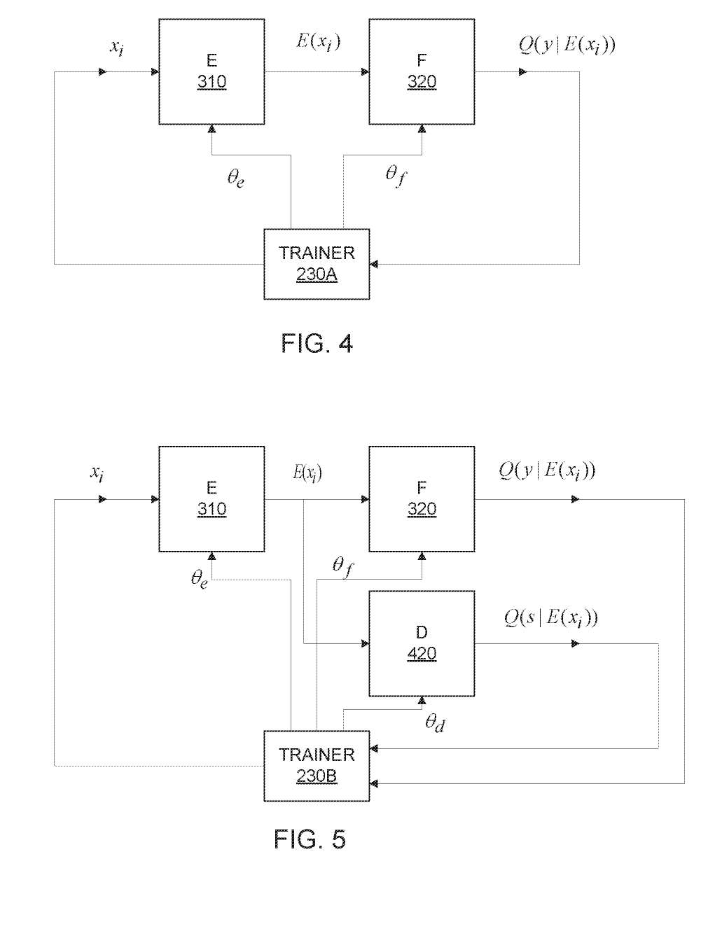

Referring to FIG. 4, one conventional approach to determining the parameters θ=(θₑ,θ_{f}) 121 is to select the parameter values to minimize a cost function (also referred to as a loss function) defined as

$${ℒ}_{f} = {\sum }_{i} {ℒ}_{f}^{i} = {\sum }_{i} - log {Q}_{F} ( {y}_{i} \vert E ( {x}_{i} ) )$$

where the sum over i is over the training observations of the training data, in which yᵢ is the “true” sleep stage, and xᵢ is the input to the sleep stage tracker 120. Note that in the approach, the source sᵢ is ignored. In this approach the parameters of the encoder E 310 and label predictor F 320 are iteratively updated by the trainer 230A (a version of trainer 230 of FIG. 2) using a gradient approach in which the parameters are updated as

$${θ}_{e} ← {θ}_{e} - {η}_{e} {∇}_{{θ}_{e}} \frac{1}{m} {\sum }_{i} {ℒ}_{f}^{i} , and$$

$${θ}_{f} ← {θ}_{f} - {η}_{f} {∇}_{{θ}_{f}} \frac{1}{m} {\sum }_{i} {ℒ}_{f}^{i} ,$$

where the sum over i is over a mini-batch of training samples of size m, and the factors ηₑ and η_{f} control the size of the updates.

Although the conventional approach may be useful in situations in which a large amount of training data is available, a first preferred approach, which is referred to as “conditional adversarial training” is used. Referring to FIG. 5, this training approach makes use of a parameterized “discriminator” D 420, which produces as output a distribution Q(s|E(xᵢ)) over possible sources s of an observation xᵢ or observation sequence xᵢ encoded by encoder E 310. The discriminator D 420 is parameterized by parameters θ_{d}, which are computed during the training process, but are not retained as part of the parameters θ 121 used by the runtime system.

It should be recognized that to the extent that the output of the discriminator D 420 successfully represents the true source, the following cost function will be low:

$${ℒ}_{d} = {\sum }_{i} {ℒ}_{d}^{i} = {\sum }_{i} - log {Q}_{D} ( {s}_{i} \vert E ( {x}_{i} ) ) .$$

Therefore, the parameters θ_{d} that best extract information characterizing the source sᵢ of each training sample minimizes [symbol] _{d}. The less information about the sources that is available from the encoded observations E(xᵢ), the greater [symbol] _{d} will be.

In this first preferred training approach, a goal is to encode the observations with the encoder E 310, such that as much information about the sleep stage is available in the output of the label predictor F 320, while as little information as feasible about the training source is available at the output of the discriminator D 420. To achieve these dual goals, a weighted cost function is defined as

[symbol]

ⁱ= [symbol] _{f}−λ [symbol] ⁱ_{d}.

and the overall cost function for each training sample is defined as

$$ν = {\sum }_{i} {ν}^{i} = {ℒ}_{f} - {λℒ}_{d} .$$

Note that the less information about the sources that is available from the encoded observations E(xᵢ), the smaller ν will be, as well as the more information about the sleep stage, the smaller ν will be.

A “min-max” training approach is used such that the parameters are selected to achieve

(θₑ,θ_{f},θ)=arg min_{θ}_{ₑ}_{,θ}_{_{f}} (max_{θ}_{_{d}} ν)=arg min_{θ}_{ₑ}_{,θdi f}( [symbol] _{f}−λmin_{θ}_{_{d}} [symbol] _{d}).

That is, for any particular choice of (θₑ,θ_{f}), the parameters θ_{d} that allows D to extract the most information about the source are selected by minimizing [symbol] _{d} over θ_{d}, and the choices of (θₑ, θ_{f}) are jointly optimized to minimize the joint cost ν= [symbol] _{f}−λ [symbol] _{d}.

This min-max procedure can be expressed in the following nested loops:

Procedure 1:

```
for a number of training iterations do
  for a mini-batch of m training triples {(xᵢ, yᵢ, sᵢ)}
    $update {θ}_{e} ← {θ}_{e} - {η}_{e} {∇}_{{θ}_{e}} \frac{1}{m} {\sum }_{i} {L}_{f}^{i} \text{;}$
    $update {θ}_{f} ← {θ}_{f} - {η}_{f} {∇}_{{θ}_{f}} \frac{1}{m} {\sum }_{i} {L}_{f}^{i} \text{;}$
    repeat
      $update {θ}_{d} ← {θ}_{d} - {η}_{d} {∇}_{{θ}_{d}} \frac{1}{m} {\sum }_{i} {L}_{d}^{i}$
    $until \frac{1}{m} {\sum }_{i} {L}_{d}^{i} < H ( s )$
  end for
end for
```

In this procedure, H(s) is the entropy defined as the expected value of −log P(s) over sources s, where P(s) is the true probability distribution of source values s, and ηₑ, η_{f}, and η_{d} are increment step sizes.

In a second preferred training approach used Procedure 1. However, an alternative discriminator D 520 takes an input in addition to E(xᵢ) that represents the information of which sleep stage is present. In particular, the second input is the true distribution P(y|xᵢ). By including this second input, the discriminator essentially removes conditional dependencies between the sleep stages and the sources. However, it should be recognized that P(y|xᵢ) may not be known, and must be approximated in some way.

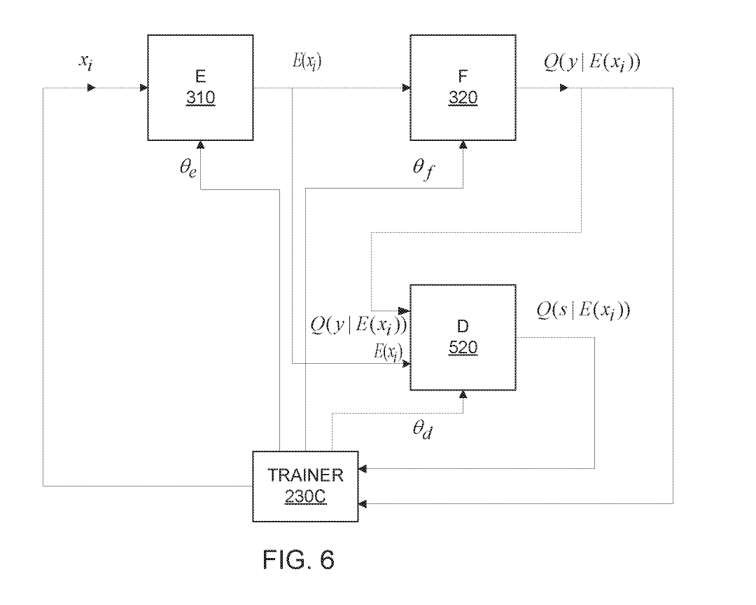

Referring to FIG. 6, a third preferred approach is similar to the second preferred approach but approximates P(y|xᵢ) using Q_{F}(y|E(xᵢ)) output from the label predictor F 320. Note that in the inner loop of updating θ_{d} according to Procedure 1, Q_{F}(y|E(xᵢ)) remains fixed. As introduced above, after completing the updating of the parameters (θₑ,θ_{f},θ_{d}) according to Procedure 1, (θₑ,θ_{f}) are retained and provided to configure the runtime system.

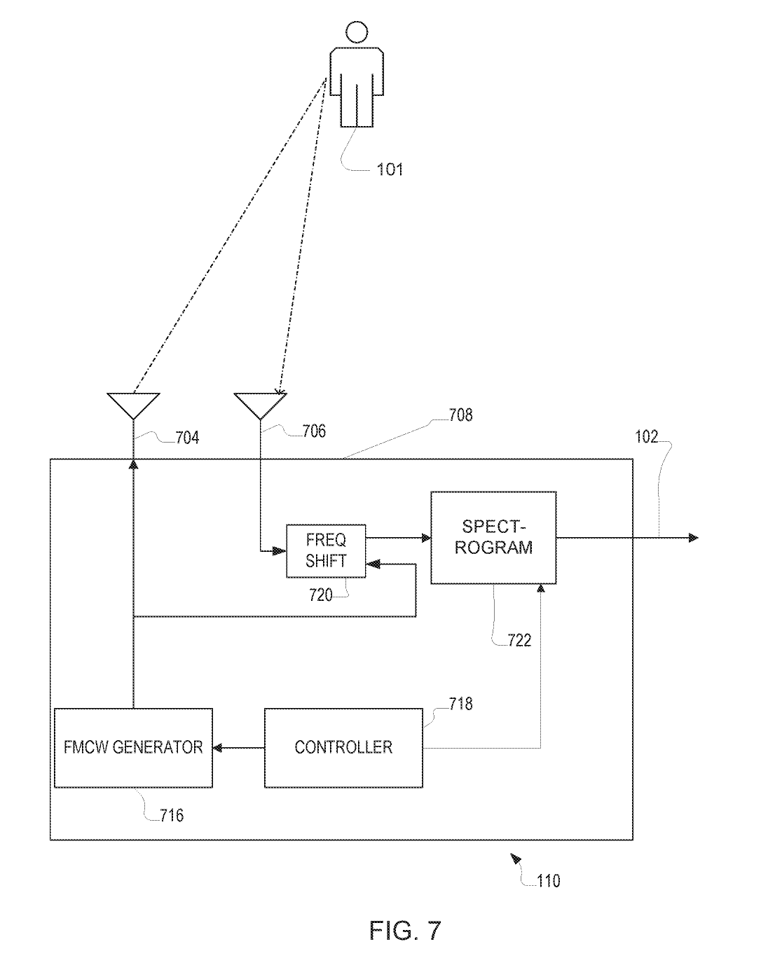

As introduced above, the signal acquisition module 110 shown in FIG. 1 provides one multi-valued observation every 30 seconds. In one embodiment, the signal acquisition system uses an approach described in U.S. Pat. Pub. 2017/0042432, titled “Vital Signs Monitoring Via Radio Reflections,” and in U.S. Pat. No. 9,753,131, titled “Motion Tracking Via Body Radio Reflections.” Referring to FIG. 7, the signal acquisition module 110 acquires signals 102 from the subject 101 without requiring any physical contact with the subject. Signal acquisition system 110 includes at least one transmitting antenna 704, at least one receiving antenna 706, and a signal processing subsystem 708. Note that, in some examples, rather than having a single receiving antenna and a single transmitting antenna, the system 100 includes a plurality of receiving antennas and/or a plurality of receiving antennas. However, for the sake of simplifying the description only to a single receiving/single transmitting antenna are shown.

In general, the signal acquisition module 110 transmits a low power wireless signal into an environment from the transmitting antenna 704. The transmitted signal reflects off of the subjects 101 (among other objects such as walls and furniture in the environment) and is then received by the receiving antenna 706. The received reflected signal is processed by the signal processing subsystem 708 to acquire a signal that includes components related to breathing, heart beating, and other body motion of the subject.

The module 110 exploits the fact that characteristics of wireless signals are affected by motion in the environment, including chest movements due to inhaling and exhaling and skin vibrations due to heartbeats. In particular, as the subject breathes and as his or her hearts beat, a distance between the antennas of the module 110 and the subject 101 varies. In some examples, the module 110 monitors the distance between the antennas of the module and the subjects using time-of-flight (TOF) (also referred to as “round-trip time”) information derived for the transmitting and receiving antennas 704, 706. In this embodiment, with a single pair of antennas, the TOF associated with the path constrains the location of the respective subject to lie on an ellipsoid defined by the three-dimensional coordinates of the transmitting and receiving antennas of the path, and the path distance determined from the TOF. Movement associated with another body that lies on a different ellipsoid (i.e., another subjects that are at different distances from the antennas) can be isolated and analyzed separately.

As is noted above, the distance on the ellipsoid for the pair of transmitting and receiving antennas varies slightly with to the subject's chest movements due to inhaling and exhaling and skin vibrations due to heartbeats. The varying distance on the path between the antennas 704, 706 and the subject is manifested in the reflected signal as a phase variation in a signal derived from the transmitted and reflected signals over time. Generally, the module generates the observation value 102 to represent phase variation from the transmitted and reflected signals at multiple propagation path lengths consistent with the location of the subject.

The signal processing subsystem 708 includes a signal generator 716, a controller 718, a frequency shifting module 720, and spectrogram module 722.

The controller 718 controls the signal generator 716 to generate repetitions of a signal pattern that is emitted from the transmitting antenna 104. The signal generator 716 is an ultra-wide band frequency modulated carrier wave (FMCW) generator 716. It should be understood that in other embodiments other signal patterns and bandwidth than those described below may be used while following other aspects of the described embodiments.

The repetitions of the signal pattern emitted from the transmitting antenna 704 reflect off of the subject 101 and other objects in the environment, and are received at the receiving antenna 706. The reflected signal received by receiving antenna 706 is provided to the frequency shifting module 720 along with the transmitted signal generated by the FMCW generator 716. The frequency shifting module 720 frequency shifts (e.g., “downconverts” or “downmixes”) the received signal according to the transmitted signal (e.g., by multiplying the signals) and transforms the frequency shifted received signal to a frequency domain representation (e.g., via a Fast Fourier Transform (FFT)) resulting in a frequency domain representation of the frequency shifted received signal. Because of the FMCW structure of the transmitted signal, a particular path length for the reflected signal corresponds to a particular FFT bin.

The frequency domain representation of the frequency shifted signal is provided to the spectrogram module which selects a number of FFT bins in the vicinity of a primary bin in which breathing and heart rate variation is found. For example, 10 FFT bins are selected in the spectrogram module 722. In this embodiment, an FFT is taken every 20 ms, and a succession of 30 seconds of such FFT are processed to produce one observation value 102 output from the signal acquisition module 110.

It should be understood that other forms of signal acquisition may be used. For example, EEG signals may be acquired with contact electrodes, breathing signals may be acquired with a chest expansion strap, etc. But it should be recognized that the particular form of the signal acquisition module does not necessitate different processing by the remainder of the sleep tracking system.

Experiments were conducted with a dataset referred to as the “RF-sleep” dataset. RF-Sleep is a dataset of RF measurements during sleep with corresponding sleep stage labels. The sleep studies are done in the bedroom of each subject. A radio device was installed in the bedroom. As described above, the signal acquisition module of the device transmits RF signals and measure their reflections while the subject is sleeping on the bed.

During the study, each subject sleeps with an FDA-approved EEG-based sleep monitor, which collects 3-channel frontal EEG. The monitor labels every 30-second of sleep with the subject's sleep stage. This system has human-level comparable accuracy.

The dataset includes 100 nights of sleep from 25 young healthy subjects (40% females). It contains over 90k 30-second epochs of RF measurements and their corresponding sleep stages provided by the EEG-based sleep monitor. Approximately 38,000 epochs of measurements have also been labeled by the sleep specialist.

Using a random split into training and validation sets (75%/25%), the inferred sleep stages were compared to the EEG-based sleep stages. The sleep stages(s) can be “Awake,” “REM,” “Light,” and “Deep.” For these four stages, the accuracy of the system was 80%.

The approach to training the system using the conditional adversarial approach, as illustrated in FIG. 6, is applicable to a wide range of situations other than in sleep tracking. That is, the notion that the cascade of an encoder (E) and a classifier (F) should be trained to match desired characteristics (e.g., the sleep stage), while explicitly ignoring known signal collection features (e.g., the subject/condition), can be applied to numerous situations in which the encoder and classifier are meant to explicitly extrapolate beyond the known signal collection features. Furthermore, although described in the context of training artificial neural networks, effectively the same approach may be used for a variety of parameterized approaches that are not specifically “neural networks.”

Aspects of the approaches described above may be implemented in software, which may include instruction stored on a non-transitory machine-readable medium. The instructions, when executed by a computer processor perform function described above. In some implementations, certain aspects may be implemented in hardware. For example the CNN or RNN may be implemented using special-purpose hardware, such as Application Specific Integrated Circuits (ASICs) of Field Programmable Gate Arrays (FPGAs). In some implementations the processing of the signal may be performed locally to the subject, while in other implementations, a remote computing server may be in data communication with a data acquisition device local to the user. In some examples the output of the sleep stage determination for a subject is provided on a display, for example, for viewing or monitoring by a medical clinician (e.g., a hospital nurse). In other examples, the determined time evolution of sleep stage is provided for further processing, for example, by a clinical diagnosis or evaluation system, or for providing report-based feedback to the subject.

It is to be understood that the foregoing description is intended to illustrate and not to limit the scope of the invention, which is defined by the scope of the appended claims. Other embodiments are within the scope of the following claims.

What is claimed is:

1. A method for tracking a sleep stage of a subject comprising:
determining a sequence of observations by sensing the subject over an observation time period;
processing the sequence of observations to yield a corresponding sequence of encoded observations, wherein the processing of the sequence of observations includes using a first artificial neural network (ANN) to process a first observation to yield a first encoded observation; and
processing the sequence of encoded observations to yield a sequence of sleep stage indicators representing sleep stage of the subject over the observation time period, including processing a plurality of the encoded observation values, which includes the first encoded observation value, using a second artificial network (ANN), to yield a first sleep stage indicator.

2. The method of claim 1 wherein each observation corresponds to at least a 30 second interval of the observation period.

3. The method of claim 1 wherein the first ANN is configured to reduce information representing a source of the sequence of observations in the encoded observations.

4. The method of claim 1 wherein the first ANN comprises a convolutional neural network (CNN).

5. The method of claim 1 wherein the second ANN comprises a recurrent neural network (RNN).

6. The method of claim 1 wherein the sequence of sleep stage indicators includes a sequence of inferred sleep stages from a predetermined set of sleep stages.

7. The method of claim 1 wherein the sequence of sleep stage indicators includes a sequence of probability distributions of sleep stage across a predetermined set of sleep stages.

8. The method of claim 1 wherein determining the sequence of observations includes acquiring a signal including at least a component representing the subject's breathing, and processing the acquired signal to produce the sequence of observations such that the observations in the sequence represent variation in the subject's breathing.

9. The method of claim 1 wherein acquiring the sequence of observation values includes emitting a radio frequency reference signal, receiving a received signal that includes a reflected signal comprising a reflection of the reference signal from the body of the subject, and processing the received signal to yield an observation value representing motion of the body of the subject during a time interval within the observation time period.

10. The method of claim 9 wherein processing the received signal includes selecting a component of the received signal corresponding to a physical region associated with the subject, and processing the component to represent motion substantially within that physical region.

11. The method of claim 1 wherein acquiring the sequence of observation values comprises acquiring signals from sensors affixed to the subject.

12. A method for tracking a sleep stage of a subject comprising:
acquiring a sequence of observations by sensing the subject over an observation time period;
processing the sequence of observations to yield a corresponding sequence of encoded observations, wherein the processing of the sequence of observations includes using a first parameterized transformation, configured with values of a first set of parameters, to process a first observation to yield a first encoded observation; and
processing the sequence of encoded observations to yield a sequence of sleep stage indicators representing sleep stage of the subject over the time period, including processing a plurality of encoded observations, which includes the first encoded observation, using a second parameterized transformation, configured with values of a second set of parameters, to yield a first sleep stage indicator;
wherein the method includes determining the firsts set of parameter values and the second set of parameter values by processing reference data that represents a plurality of associations, each association including an observation, a corresponding sleep stage), and a corresponding source value, wherein the processing determines values of the first set of parameters to optimize a criterion to increase information in the encoded observations, determined from an observation according to the values of the first set of parameters, related to corresponding sleep stages, and to reduce information in the encoded observations related to corresponding source values.

13. The method of claim 12 wherein processing the reference data that represents a plurality of associations further includes determining values of a third set of parameters associated with a third parameterized transformation, third parameterized transformation being configured to process an encoded observation to yield and indicator of a source value.

14. The method of claim 13 wherein the processing of the reference data determines values of the first set of parameters, values of the second set of parameters, and values of the third set of parameters to optimize the criterion.

15. The method of claim 14 wherein information in the encoded observations related to corresponding sleep stages depends on the values of the second set of parameter and information in the encoded observation values related to corresponding source values depends on the values of the third set of parameters.

16. A machine-readable medium comprising instructions stored thereon, which when executed by a processor cause the processor to:
determining a sequence of observations resulting from sensing a subject over an observation time period;
processing the sequence of observations to yield a corresponding sequence of encoded observations, wherein the processing of the sequence of observations includes using a first artificial neural network (ANN) to process a first observation to yield a first encoded observation; and
processing the sequence of encoded observations to yield a sequence of sleep stage indicators representing sleep stage of the subject over the observation time period, including processing a plurality of the encoded observation values, which includes the first encoded observation value, using a second artificial network (ANN), to yield a first sleep stage indicator.

17. A sleep tracker comprising:
a signal acquisition system, configured to determining a sequence of observations resulting from sensing a subject over an observation time period; and
a tracker comprising an encoder and a label predictor, wherein the encoder implements a parameterized transformation of the observations to form encoded observations according to stored parameters selected to reduce information representing a source of the sequence of observations in the encoded observations, and wherein the label predictor implements a parameterized transformation of the encoded observations to yield sleep stage indicators representing sleep stage of the subject over the observation time period.
